(19) United States
(12) Patent Application Publication
(10) Pub. No.: US 2008/0075701 A1
(43) Pub. Date: Mar. 27, 2008

(54) Composition for magnetofection

(76) Inventors: Chin-Yih Rex Hong, Taipei (TW); Herng-Er Horng, Taipei (TW); Jui-Sheng Sun, Taipei City (TW); Hong-Chang Yang, Taipei (TW); Shieh-Yueh Yang, Sindian City (TW)

Correspondence Address: J.C. Patents, Suite 250, 4 Venture, Irvine, CA, 92618, US

(21) Appl. No.: 11/525,333

(22) Filed: Sep. 21, 2006

(51) Int. Cl. A61K 48/00; C12N 15/88

(52) U.S. Cl. 424/93.21; 435/458

(57) ABSTRACT

A composition for magnetofecting genetic materials into cells with the assistant of an external magnetic force is described. The composition includes hydrophilic vectors, magnetic nanoparticles, and genetic materials, wherein the magnetic nanoparticles and the genetic materials are enveloped inside the hydrophilic vectors.

### BACKGROUND OF THE INVENTION

1. Field of Invention

The present invention relates to a composition for delivering molecules into cells. More particularly, the present invention relates to a composition for the magnetofection of genetic materials using magnetic nanoparticles.

2. Description of Related Art

The delivery of genetic materials into target cells is a powerful tool to manipulate or modify the activities of nucleic acids in the cells. One conventional technology for the delivery of genetic materials is electroporation. Electroporation is accomplished by an introduction of a quick voltage shock to temporarily disrupt areas of the phospholipid bilayer of the plasma membrane of the cell. The creation of the transient pores in the membrane allows molecules to pass through. Then, the membrane is reassembled spontaneously and the cell remains intact. The electroporation method, however, has two main fatal disadvantages: low delivery rate and low cell survival rate.

To improve these disadvantages, virus has been proposed to serve as a vector to deliver genetic materials to target cells. Although viral transfection has a higher efficiency in delivering genetic materials, numerous problems still exists, for example, cytotoxicity. Accordingly, the development of a nontoxic and highly-efficient method is being intensively pursued.

One of the promising methods for delivering genetic materials into target cells is magnetofection. Magentofection is a delivery method of genetic materials, wherein the genetic materials are associated with magnetic particles coated with cationic molecules. The magnetic particles-genetic materials complexes are then transported into cells under the influence of an external magnetic field. Many reports have demonstrated that magnetofection is nontoxic, highly efficient and versatile. Therefore, mangetofection is gradually becoming a main-trend method for delivering genetic materials molecules into cells.

Typically, the magnetic particles-genetic materials complexes are formed by binding the gene vectors to the surfactants coated on the magnetic particles. Accordingly, the genetic materials are exposed in the solution during the delivery process. The exposure of the genetic materials in the solution may lead to damages to the genetic materials. Consequently, the delivery efficiency is reduced.

### SUMMARY OF THE INVENTION

In view of the foregoing, the present invention provides a composition and a method for synthesizing the composition for magnetofection of genetic materials, wherein the delivery efficiency of genetic materials to a cell is enhanced.

The present invention also provides a composition and a method for synthesizing the composition for magnetofection of genetic materials, wherein the magnetic particles-genetic materials complex is enveloped inside a vector. Consequently, the genetic materials are prevented from being damaged.

As embodied and broadly described herein, a composition for magnetofecting genetic materials into cells includes hydrophilic vectors, which are vesicles having a hydrophilic exterior. The composition further includes magnetic nanoparticles and transfected genetic materials, wherein the magnetic nanoparticles and the transfected genetic materials are enveloped inside the hydrophilic vectors. The hydrophilic vectors are, for example, liposomes. In one embodiment, the liposomes are created with lipid chains, which include but not limited to 1,2-dioleoyl-3-trimethylammonium-propane and 1,2-dioleoyl-3-sn-phosphatidyl-ethanolamine.

According to a method for synthesizing the composition for the magnetofection of genetic materials, hydrophilic vectors in an aqueous solution are provided. Magnetic nanoparticles that are originally in the water-based magnetic fluid are added to the hydrophilic vector aqueous solution, wherein some of the magnetic nanoparticles are enveloped within the hydrophilic vectors. The magnetic nanoparticles are originally coated with organic acid, which is removed during the enveloping process. The organic acid includes, but not limited to, lauric acid, oleic acid, etc. Thereafter, transfected genetic materials are added into the solution having the magnetic nanoparticles encapsulated by the hydrophilic vectors. The transfected genetic materials are transported through the hydrophilic vectors to couple with the magnetic nanoparticles therein to form a genetic materials-magnetic particles complex.

Since the genetic materials are encapsulated inside the hydrophilic vectors, they are precluded from being damaged during the delivery process. Hence, the delivery efficiency is enhanced.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. The patent or application file contains at least one drawing executed in color. Copies of this patent or patent application publication with color drawing(s) will be provided by the Office upon request and payment of the necessary fee.

FIG. 1 is a schematic diagram illustrating the composition for magnetofection of genetic materials according to one embodiment of the invention.

FIG. 2 is a flow chart of steps in exemplary processes that may be used in the synthesis of the composition for magnetofection of genetic materials in accordance to one embodiment of the invention.

FIGS. 3a to 3c are the laser scattering analysis results respectively showing the size distributions of the liposome shell, the liposomes with magnetic nanoparticles enveloped therein, and the liposomes with genetic materials-magnetic nanoparticle complexes enveloped therein, and the genetic materials-magnetic nanoparticle complexes are synthesized according to the process shown in FIG. 2.

FIG. 4 is a schematic diagram illustrating the operation of magnetofection with and without the influence of an external magnetic field.

FIG. 5 shows images of the mouse osteoblast cells before and after magnetofection using the composition of the invention.

### DESCRIPTION OF THE PREFERRED EMBODIMENTS

Synthesis of Genetic Materials-Magnetic Particles Composition

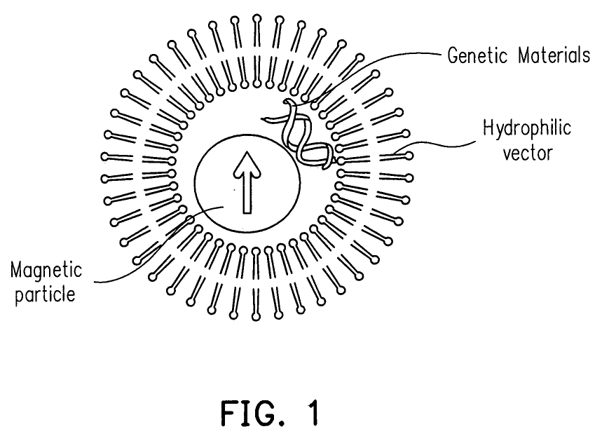

FIG. 1 is a schematic diagram illustrating the composition for magnetofection of genetic materials according to one embodiment of the invention. As shown in FIG. 1, the composition for the magnetofection of genetic materials includes at least a hydrophilic vector, magnetic nanoparticles and genetic materials, wherein the magnetic nanoparticles and the genetic materials are encapsulated inside the hydrophilic vector. In this embodiment, the hydrophilic vector is a liposome, which is a spherical vesicle with a membrane composed of a phospholipid bilayer. In one embodiment, the liposome is created with lipid chains include but not limited to 1,2-dioleoyl-3-trimethylammonium-propane (DOTAP) and 1,2-dioleoyl-3-sn-phosphatidyl-ethanolamine (DOPE). It is appreciated that the liposomes can also be created from a variety of artificial and biological lipids. The genetic materials includes, for example, gene, nucleic acid such as DNA (Deoxyribonucleic acid) and RNA (Ribonucleic acid).

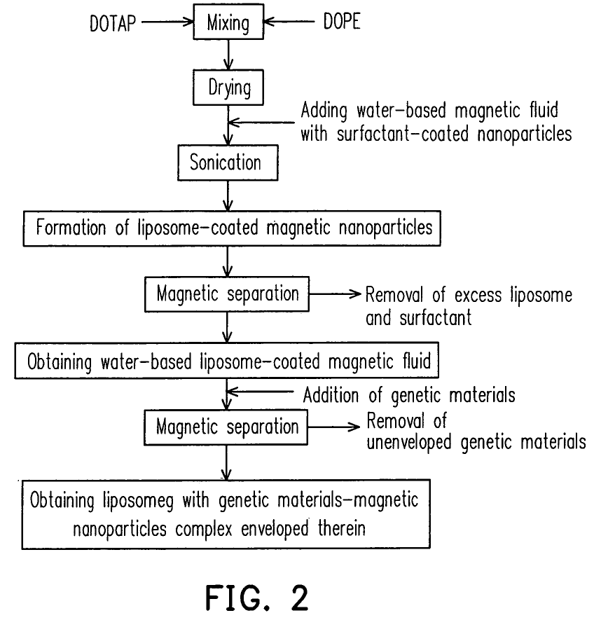

FIG. 2 is a flow chart of steps in exemplary processes that may be used in the synthesis of the composition for magnetofection of genetic materials. As shown in FIG. 2, the synthesis of the composition for magnetofection of genetic materials according to one embodiment of the invention is accomplished by first mixing the lipid chains in an organic solution and dehydrating the phospholipid film. The lipid chains can be, but not limited to DOTAP and DOPE. The dehydrated phospholipid film (DOTAP:DOPE, hereinafter referred as liposomes) is added into a water-based magnetic fluid and the resulting solution is sonicated. The magnetic fluid contains magnetic nanoparticles that are coated with surfactant, for examples, organic acids including but not limited to lauric acid, oleic acid, etc. The material of the magnetic nanoparticles includes but not limited to Fe₃O₄, Fe₂O₃, MnFe₃O₄, NiFeO₄ and CoFe₂O₄. Subsequent to the sonication process, the magnetic nanoparticles become uncoated from the surfactant and are encapsulated within the liposomes, leaving the surfactant coating outside the liposome capsulse. In essence, during the encapsulation process, the surfactant is separated from the magnetic nanoparticles and is not enveloped by the hydrophilic vectors. The surfactant and the liposomes without magnetic particles enveloped therein are removed through a magnetic separation process.

Thereafter, genetic materials, such as nucleic acid, transfected gene or DNA/RNA molecules are added into the solution having the liposome-coated magnetic nanoparticles. The genetic materials can be transported through the liposome shell to join the magnetic nanoparticles therein. The un-enveloped genetic materials can be separated from the genetic materials-magnetic particles complex enveloped inside the liposomes via magnetic separation.

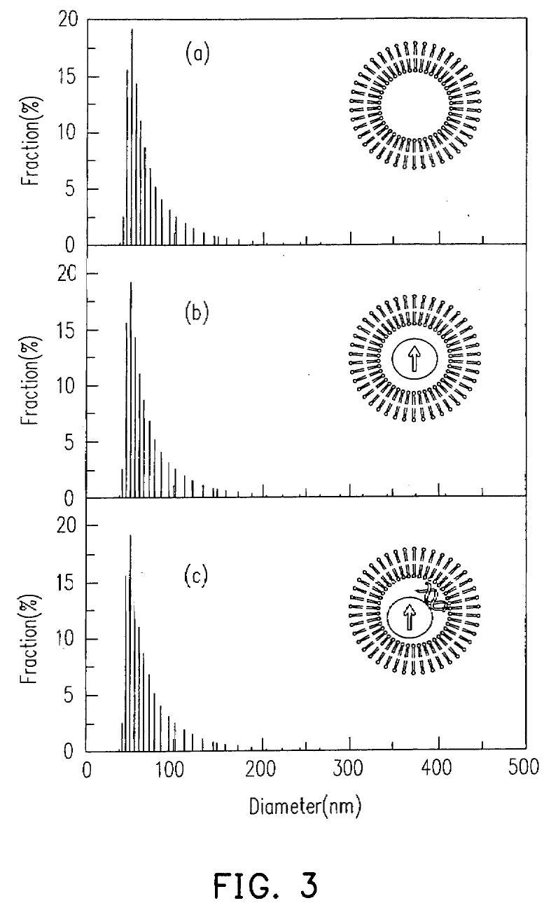

In order to be applicable in the magnetofection of genetic materials, the liposomes having the genetic materials-magnetic nanoparticles complex enveloped therein can not be too large. The size of the complex is preferably smaller than 100 nm. The size distribution of the liposomes having the genetic materials-magnetic nanoparticles complex enveloped therein is measured by using laser scattering analysis and the results are shown in FIGS. 3a to 3c. The liposomes having the genetic materials-magnetic nanoparticles complex enveloped therein are synthesized via the process shown in FIG. 2. As shown in FIG. 3a, the average diameter of the liposome shell is about 64.0±8.4 nm. As shown in FIG. 3b, the average diameter of the liposomes with magnetic nanoparticles having a mean diameter around 25 nm inserted inside the liposome shell is about 66.2±9.5 nm. The average diameter of the liposomes with the genetic materials-magnetic nanoparticles complex enveloped therein is about 67.8±9.3 nm as shown in FIG. 3c. These results reveal that the complex is small enough for magnetofection. Further, the liposomes with the complex enveloped therein are almost the same size as the original liposome shell. Hence, the laser scattering analysis results confirm that the magnetic nanoparticles and the genetic materials are enveloped inside the liposomes. In the case that the genetic materials are bound to the outer surface of the liposome shell, the size of the complex would be much larger than that of the liposome shell.

Operation of In-vitro Magnetofection

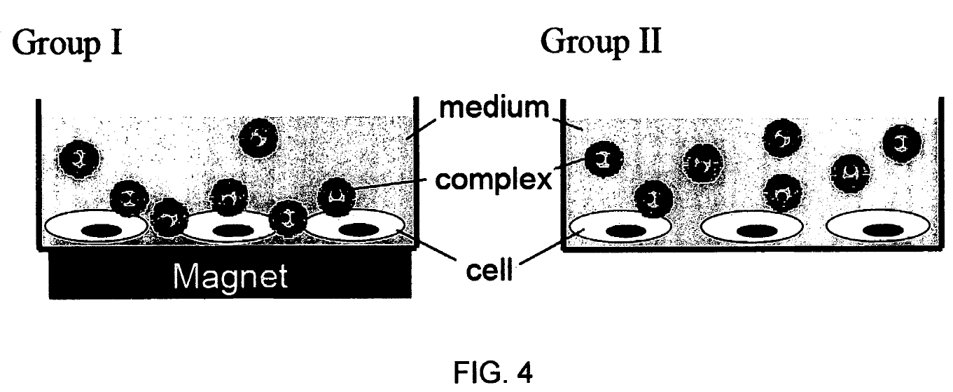

FIG. 4 is a schematic diagram illustrating the operation of magnetofection with and without the influence of an external magnetic force. As schematically illustrated in FIG. 4, equal amount of cells are laid at the bottoms of culture wells and are submerged with cultured medium. Each culture well is added with the same tiny amount of the liposomes having the genetic materials-magnetic nanoparticles complex enveloped therein. These wells are then divided into two groups, wherein Group I is positioned above magnets, while Group II is subjected to zero magnetic field. After adding the liposomes into the wells for a certain period of time, the activity of mangetofection is investigated.

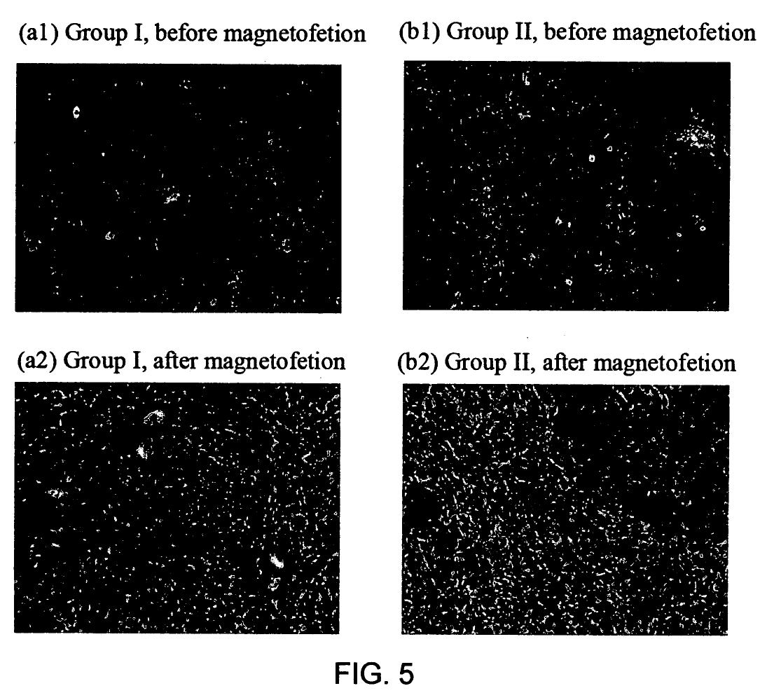

The following disclosure is an example of magnetofecting gene Lac Z into osteoblast cells of mice. The magnetic nanoparticles used in this example are Fe₃O₄. FIGS. 5(a1) and 5(b1) respectively show the images of cells at the bottoms of Group I wells and Group II wells before magnetofection.

To achieve magnetofection, 80 μl of the liposome solution having about 1 μg of Lac Z is added into the cells shown in FIGS. 5(a1) and 5(b1). After incubating the cells for 10 days, the images of cells were again taken and shown in FIGS. 5(a2) and 5(b2). The blue cells denote Lac Z have been successfully transfected into the cells. It is worthy to note that Group I exhibits a much higher efficiency for magnetofecting Lac Z into cells because more cells become blue in FIG. 5(a2) as compared to FIG. 5(b2). In other words, with the assistance of magnetic force, the efficiency of magnetofecting gene/DANA molecules into cells is enhanced.

The results further support that the composition of the invention is non-toxic and is effective in the delivery of desired genetic/DNA materials into the target cells.

Moreover, since the genetic materials are enveloped within the liposomes, they are well protected from being damaged during the delivery process. Hence, the delivery efficiency is enhanced.

It will be apparent to those skilled in the art that various modifications and variations can be made to the structure of the present invention without departing from the scope or spirit of the invention. In view of the foregoing descriptions, it is intended that the present invention covers modifications and variations of this invention if they fall within the scope of the following claims and their equivalents.

1. A composition for magnetofecting cells, the composition comprising:
hydrophilic vectors, wherein each hydrophilic vector is a vesicle having a hydrophilic exterior;
magnetic nanoparticles; and
genetic materials, wherein the magnetic nanoparticles and the genetic materials are enveloped inside the hydrophilic vectors.

2. The composition of claim 1, wherein the hydrophilic vectors are liposomes.

3. The composition of claim 2, wherein the liposomes are formed with lipid chains comprising at least 1,2-dioleoyl-3-trimethylammonium-propane (DOTAP) and 1,2-dioleoyl-3-sn-phosphatidyl-ethanolamine (DOPE).

4. The composition of claim 1, wherein a material of the magnetic nanoparticles comprises at least one of Fe₃O₄, Fe₂O₃, MnFe₃O₄, NiFeO₄ and CoFe₂O₄.

5. The composition of claim 1, wherein the genetic materials comprise at least one of nucleic acids, transfected gene, DNA (Deoxyribonucleic acid) molecules and RNA (ribonuecleic acid) molecules.

6-13. (canceled)
